(19) United States
(12) Patent Application Publication
(10) Pub. No.: US 2013/0196343 A1
(43) Pub. Date: Aug. 1, 2013

(54) SIGNAL LOCKING LABEL FREE BIOSENSING

(71) Applicants: Carlos H. Mastrangelo, Salt Lake City, UT (US); Layne Daryl Williams, Salt Lake City, UT (US)

(72) Inventors: Carlos H. Mastrangelo, Salt Lake City, UT (US); Layne Daryl Williams, Salt Lake City, UT (US)

(21) Appl. No.: 13/636,638

(22) PCT Filed: Mar. 31, 2011
(86) PCT No.: PCT/US11/30773

§ 371 Date: Feb. 21, 2013

Related U.S. Application Data
(60) Provisional application No. 61/319,668, filed on Mar. 31, 2010.
(60) Provisional application No. 61/353,153, filed on Jun. 9, 2010.

(51) Int. Cl. G01N 33/557; G01N 33/573

(52) U.S. Cl. CPC G01N 33/557; G01N 33/573
USPC 435/7.4; 436/501; 435/288.7; 422/69

(57) ABSTRACT

A biosensor can include a fluid flow channel (12), a pulsing mechanism (14), and a binding response measurement mechanism (16). The fluid flow channel (12) can include an inlet (18) to accept a fluid into the fluid flow channel and an outlet (20). At least one binding sensor surface (22) can be oriented within the fluid flow channel. The binding sensor surface (22) can include a fixed binding moiety on the binding sensor surface selected to bind with a complimentary target agent within the fluid to form a complimentary bound duplex. The pulsing and flow switching mechanism (14) can be configured to drive the fluid into the fluid flow channel (12) in a pulsed analyte flow.

### RELATED APPLICATIONS

This application claims priority to U.S. Provisional Application No. 61/319,668, filed Mar. 31, 2010 and U.S. Provisional Application No. 61/353,153, filed Jun. 9, 2010, each of which are incorporated herein by reference.

### GOVERNMENT INTEREST

This invention was made with government support under Grant No. ECCS-0924808 awarded by the National Science Foundation. The United States government has certain rights to this invention.

### BACKGROUND

Surface plasmon resonance (SPR) is a popular technique for label-free detection of biomolecular interactions at a surface. SPR yields quantitative kinetic association and dissociation constants of surface interactions such as the binding of two molecular species, one present in the liquid phase and the other immobilized at the surface. Current state-of-the-art SPR systems extract kinetic constants from measurements of the step response of the interaction versus time. The step response measurement is subject to the influence of noise and drift disturbances that limit its minimum detectable mass changes. A number of techniques have been used to reduce the noise and drifts in these systems in order to detect smaller mass changes. These include the use of multiple referencing points, temperature compensation and a multiplicity of optical detectors. However, challenges remain which limit the accuracy and sensitivity of these methods.

### SUMMARY

Use of vector space signal-locking methodologies can remove much of the noise in surface plasmon resonance systems. More specifically, microfluidic chemical signal generator chips are used to provide complex fluid input signals on a label free biosensing system such as a surface plasmon resonance imaging (SPRi) system. The SPRi output is analyzed using signal-locking and Fourier methods, which in combination with the complex input signals can have the ability to reject inherent noise of the biosensor system and enable a lower limit of detection than current commercial systems can achieve.

A biosensor can include a fluid flow channel, a pulsing mechanism, and a surface binding response measurement mechanism. The fluid flow channel can include an inlet to accept a fluid into the fluid flow channel, a binding sensor surface within the fluid flow channel including a fixed binding moiety on the binding sensor surface selected to bind with a complimentary target agent within the fluid to form a complimentary bound duplex, and an outlet. The pulsing and flow switching mechanism can be configured to drive the fluid into the fluid flow channel in a pulsed analyte, constant velocity flow. The binding response measurement mechanism can be associated with the binding sensor surface to measure responses from the binding sensor surface (e.g. SPR or other response).

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1A is a diagram of a biosensor in accordance with one aspect of the invention.

FIG. 1B is a diagram showing the association/dissociation reaction on the sensor surface.

FIG. 1C is a sensorgram response, showing the association and dissociation phases.

FIG. 2A is a diagram of FT-SPR detection scheme. The input is a periodic cycle of association and dissociation steps determined by clocks φ and φ̅,

FIG. 2B is a graph frequency as a function of power illustrating the discrete frequency f_{φ} can then be discriminated from the broadband noise in the power spectra of the sensorgram output waveform.

FIG. 2C is a diagram of a FT-SPR detection scheme with dual modulators, to avoid problems associated with non-uniform analyte distributions along the channel width.

FIG. 3A is a plot of single step response data for each concentration and the associated fit.

FIG. 3B is a graph showing the resulting value for k_{obs} plotted against the respective concentration and a linear fit used to find kₐ and k_{d}.

FIG. 4A is a graph of input clock frequencies as a function of time.

FIG. 4B is a resultant sensorgram waveform from the SPR system.

FIG. 5A is a graph showing the magnitude of the normalized transfer function (Bode plot) vs. frequency.

FIG. 5B is a graph of the resulting pole frequencies vs. the respective CA-II input concentration.

FIG. 6A is a Power Density plot showing the signal (from the lowest applied concentration of CA-II) analyzed with the FT method and the system noise.

FIG. 6B is a plot of the signal to noise ratio (SNR) for each of the two methods. The experimental SNR of the FT method shows a 20 dB increase at the pole frequency over the single step method, which translates to a 100-fold improvement.

FIG. 7A is a photograph of multisine SPR chip with dual chemical signal generators and flow cells (reference and sensing).

FIG. 7B is a close up of a channel section showing the SPR sensing spots. The dual channel is used to extract the binding signal from the bulk response observed on the reference channel.

FIG. 8 is a flow diagram of a multisine SPR technique. Unlike the conventional SPR, the excitation signal spectra is narrow band. A chemical signal generator is used to generate an analyte concentration signal with a discrete number of excitation frequencies (using a multisine expansion). The spectrum of the discrete excitation frequencies is flat. Because of the binding reaction at the functionalized Au SPR spot, some of the excitation analyte reacts at the surface with characteristic association and dissociation dynamics and the SPR spectra is no longer flat and shaped by the dynamics of the reaction. The pole of the spectrum is related to the kinetic constants of the reaction.

FIG. 9A is measured time domain and FIG. 9B is the corresponding spectra of a synthesized 28,000 plug multisine chemical signal by fluorescence emission using fluorescein as a tracer. Note the discrete peaks of the multisine components. FIG. 9C is a time-domain plot of this analog signal.

FIG. 10A is measured time domain and FIG. 10B is corresponding spectra of a 28,000 plug multisine chemical signal using ethanol as a tracer in the SPR system. Again, note the discrete peaks of the multisine components.

FIGS. 11A and 11B are a comparison of SPR spectra under multisine excitation of 16 μM CA-II analyte in PBS buffer on functionalized PEG reference (FIG. 11A) and ABS sensing spots (FIG. 11B). The non-reacting reference spectrum is flat, while the reactive sensor surface shapes the spectra with a characteristic pole. The pole frequency is related to the kinetic constants of the biochemical reaction. The measurement was carried out in 16 minutes under a flow velocity of 2 cm/s.

FIG. 12A is a normalized bode plot at three different average concentrations.

FIG. 12B is a plot of pole frequency versus analyte concentration from which kₐ and k_{d} can be extracted.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### DEFINITIONS

In describing and claiming the present invention, the following terminology will be used.

The singular forms “a,” “an,” and “the” include plural referents unless the context clearly dictates otherwise. Thus, for example, reference to “a sensor spot” includes reference to one or more of such materials and reference to “subjecting” refers to one or more such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Fourier-Transform Biosensing

Microfluidic chemical signal generator chips are used to provide complex fluid input signals on a surface plasmon resonance imaging (SPRi) system. The SPRi output is analyzed using signal-locking and Fourier methods, which in combination with the complex input signals have the ability to reject the inherent noise of the biosensor system and enable a lower limit of detection than current commercial systems can achieve. The FT-SPR biosensor can reduce the minimum detectable mass for surface plasmon resonance systems, from approximately 100 Da to 10 Da. Reduction of the minimum detectable mass can enable more reliable detection of small molecules in drug discovery applications, for example.

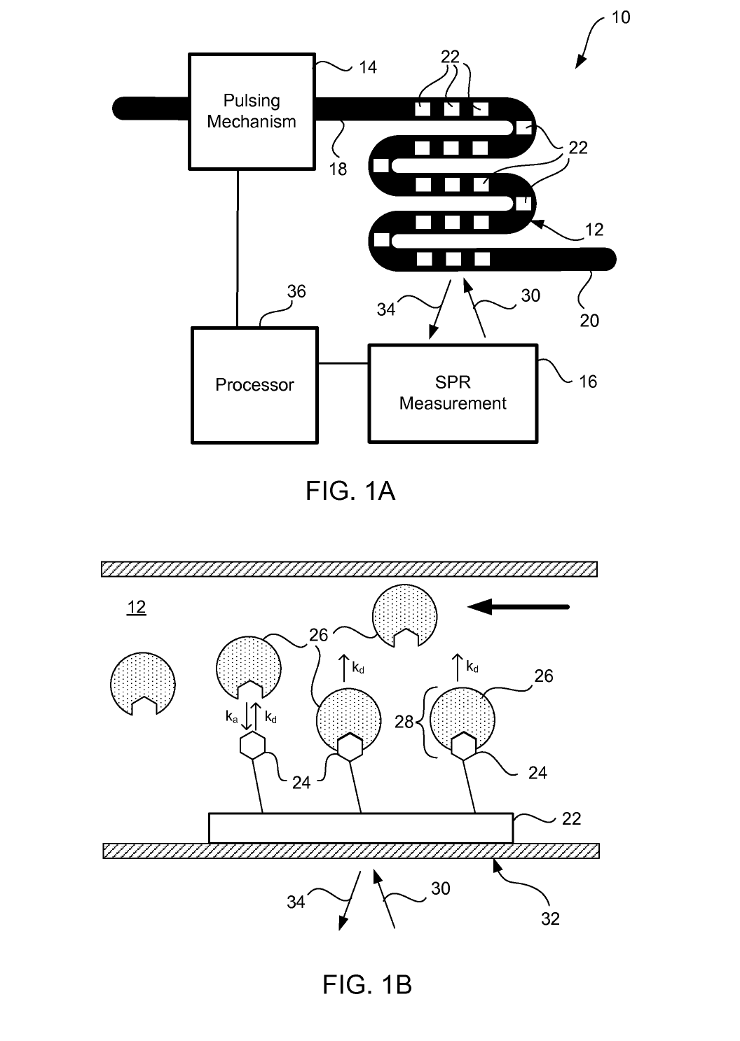

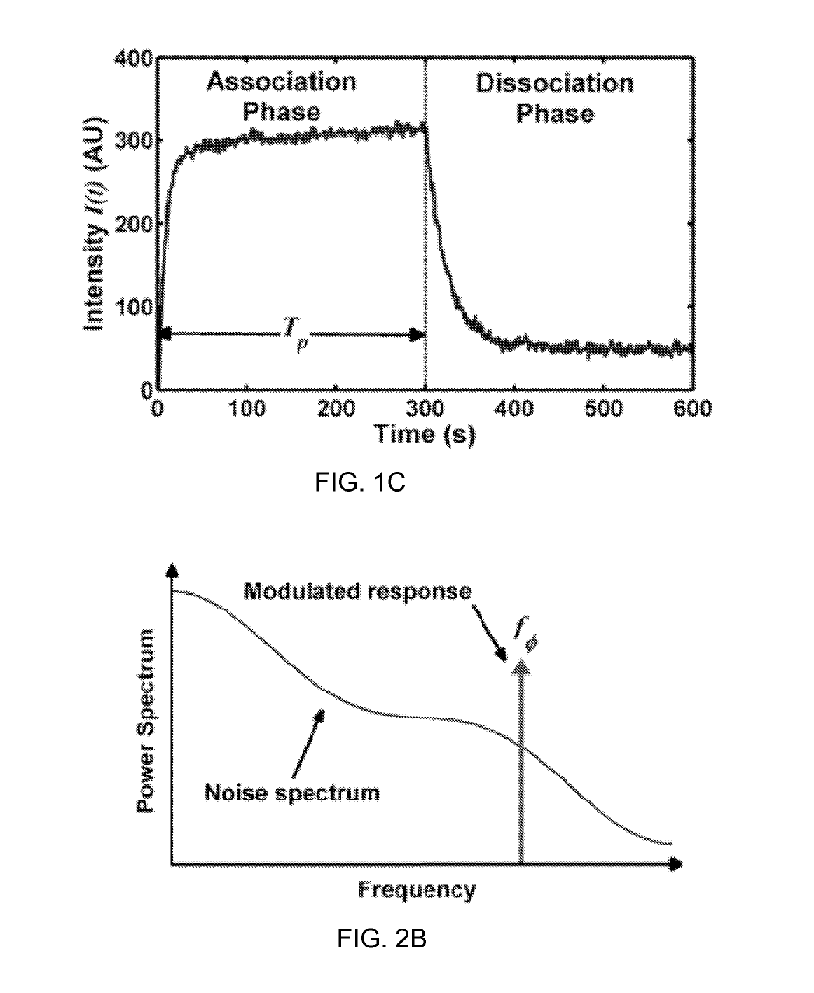

As illustrated in FIG. 1A, a biosensor 10 can include a fluid flow channel 12, a pulsing mechanism 14, and a binding response measurement mechanism 16. The fluid flow channel 12 can include an inlet 18 to accept a fluid into the fluid flow channel and an outlet 20. At least one binding sensor surface 22 can be oriented within the fluid flow channel. FIG. 1B illustrates an expanded view of a binding sensor surface 22 within the fluid flow channel. The binding sensor surface 22 can include a fixed binding moiety 24 on the binding sensor surface selected to bind with a complimentary target agent 26 within the fluid to form a complimentary bound duplex 28. Referring again to FIG. 1A, the pulsing and flow switching mechanism 14 can be configured to drive the fluid into the fluid flow channel 12 in a pulsed analyte flow. Most often the pulsed flow of analyte can be provided in a constant velocity flow. By constant velocity flow, it is meant that the flow velocity is maintained substantially constant while analyte concentration is varied. Variation of bulk velocity introduces additional complexities in data analysis, although such may be accounted for during data analysis.

The binding response measurement mechanism 16 can be associated with the binding sensor surface 22 (or surfaces) to measure responses associated with binding and release of the target agent from the binding sensor surface. Although surface plasmon resonance measurement can provide several advantages, other label free biosensing systems which can be suitable include optical and/or mechanical systems such as, but not limited to, optical resonators (e.g. SRU-BIND, optical ring resonators, etc.), optical waveguide systems, optical fiber systems, photonic crystal systems, ellipsometric and interferometric biolayer measurement (e.g. Forte Bio Octet), oscillator mass loading (e.g. q-Sense® QCD-M), mechanical deflection (e.g. due to induced stress and bending from binding), calorimetric, surface acoustic wave measurements, electrochemical responses, interferometer (e.g. Mach-Zender, Young's, Hartman, backscattering, etc.), and the like. As illustrated in FIG. 1B, an SPR measurement mechanism can typically be oriented to direct light 30 to a back side 32 of the sensor surface 22 opposite the binding surface. Reflections 34 are collected and analyzed as discussed in greater detail below.

The fluid flow channel 12 can be a microfluidic channel. Optionally, the biosensor can be embedded as part of a microfluidic chip (e.g. lab-on-a-chip). The fluid flow channel can have a channel length which avoids excessive dispersion between pulsed fluid charges. Although channel lengths can vary, a channel length of about 0.1 mm to about 10 cm can be broadly suitable. The length of the fluid-flow channel under which this technique can be used is a function of many geometrical and physical factors which determine the amount of dispersion. The channel length and velocity affects cutoff frequency (i.e. beyond which amplitude attenuation is unacceptably high). Although discussed in a different technical application, these common specific considerations are discussed in more detail in Fourier Microfluidics, Lab on a Chip, Y. Xie et al, 2008, 8, 779-785 which is incorporated herein by reference. This relationship with frequency tends to be beneficial with binding reactions are slow, while faster reactions can be accommodated by providing accordingly shorter channel lengths and dimensions. The length of the channel can be selected to produce sufficient smoothing of the waveform to allow analysis thereof as outlined in more detail below. Similarly, the height and width of the channel can be chosen to minimize dispersion while maintaining a sufficiently high velocity to prevent transport-induced false signals.

The fixed binding moiety can be any group which can be attached to the binding sensor surface and which binds to a corresponding target agent. Non-limiting examples of fixed binding moieties can include ligand, protein, DNA, RNA, membrane and combinations thereof. One specific example of a fixed binding moiety is 4-(2-aminoethyl)benzenesulfonamide. Other non-limiting examples of fixed binding moieties include polypeptides, bovine serum albumin, and the like. These binding moieties can be fixed using any suitable chemistry such as, but not limited to, amine coupling, PEGylation, and the like.

Correspondingly, non-limiting examples of the complimentary target agent can include ligand, protein, DNA, membrane and combinations thereof. One example of the complimentary target agent is carbonic anhydrase II. Other non-limiting examples of complimentary target agents can include proteins tagged with hexahistidine, glutathione transferase, proteases, kinases, drugs, and the like. As a general rule, the target agent can be a small compound having a mass less than 500 Da, in some cases 300 Da or less, and in other cases 100 Da or less.

The binding surface can be any substrate material which allows attachment of the desired fixed binding moiety to the surface. For SPR based mechanisms, the binding surface can often be gold, although other non-limiting examples of suitable materials can include Dextran. Other binding response measurement mechanisms can use gold, polymers (e.g. ABS, PE, PP, etc), composites, or other materials as the binding surface. Generally, materials which allows for binding, do not substantially degrade during use, and allow function of the binding sensing mechanism can be used as the binding surface of the sensor.

The pulsing mechanism can be provided by any mechanism which allows for pulsed flow of the analyte. One avenue for achieving this is by switching input of fluid into the sensor channel (i.e. a carrier fluid and an analyte-containing fluid). This can include the use of valves, pumps and/or baffles which control flow. In one aspect, the pulsing mechanism can include at least one fast micro fluidic valve. In another aspect, the pulsing mechanism includes two interleaved multi-plug pulse encoded modulators. One specific example of such modulators is described more fully in F. Azizi and C. H. Mastrangelo, “High-Speed Chemical Signal Generation with Multi-Plug Modulators,” MicroTAS 2008, San Diego, October 2008, pp. 84-86, which is incorporated herein by reference. Although not required, the switched analyte flow is often a substantially constant flow rate with the analyte concentration being varied with time. The corresponding switched flow can generally be pulsed at a fairly high frequency. Although various frequencies can be suitable, a general frequency range can typically be from 1 mHz to about 10 Hz is pulsed (depending on the compound to be tested) with typical frequency of 2 mHz to 250 mHz for the test example provided below. The pulsing can be a regular sinusoidal excitation. However, the frequency can be variable such that other linear transforms can be suitable. Non-limiting examples of such linear transforms can include chirp and wavelet transforms. In one specific aspect, the switched analyte flow can be multiline in analyte concentration. Typically, flow in the channel can be maintained in a laminar plug flow.

The surface plasmon resonance measurement mechanism can include a light source having adjustable incidence angle against a back side of the binding sensor surface and a light detector positioned to receive reflected light from the back side. Although other detectors can be used, the light detector can be a CCD camera. A Fourier transform module can be included for processing the surface plasmon responses in frequency domain. This can be provided by a suitable processor 36 (FIG. 1A) such as a CPU, programmable microprocessor chip, or the like. An analysis module can be configured to convert the surface plasmon responses to at least one binding property of the fixed binding moiety and the complimentary target agent. The at least one binding property includes kinetic constants (kₐ and k_{d}, for example), concentration assay, and equilibrium constants. The measurable binding property can also include discriminating these values for multiple target compounds if the variables are sufficiently different to statistically differentiate. Thus, optionally, the sensor surface can include multiple different binding moieties or different sensor surfaces can have different binding moieties fixed thereto.

In accordance with the above discussion, a biosensing method can include passing a fluid across a binding sensor surface. The fluid can include a switched analyte flow such that analyte (i.e. complimentary target agent) concentration within the fluid varies with time. The binding sensor surface can include a fixed binding moiety and the fluid includes the complimentary target agent. Binding responses such as surface plasmon resonance responses can be measured during contact of the fluid with the binding sensor surface. The binding responses can be analyzed using frequency domain analysis to identify at least one binding property between the fixed binding moiety and the complimentary target agent.

Measurement can be based on resonant angle, resonant frequency and/or polarization. Pulsing can be feedback locked using the binding responses. These methods can be used to achieve a biosensor having a minimum detectable mass of 10 Da or less.

For reactions which are very slow (i.e. a low k_{d}), additional steps can improve results. For example, instead of cycling one compound on and off, surface regeneration can be performed. In particular, a pristine sensor surface can be exposed to the compound once, and then flushed with another compound that desorbs the compound (e.g. a weak acid) to regenerate the pristine or unbound surface. In this manner, kₐ (independent of k_{d}) can be measured by repeating the exposure and regeneration cycles at varying frequencies and applying the same Fourier scheme described herein.

This biosensor measures the biomolecular interaction not in time, but over a very narrow frequency range under periodic excitation. The measured response is thus locked to a very specific narrow band signal. This narrow band spectral sensing scheme has a very high degree of rejection to uncorrelated spurious signals.

A signal-locked SPR example was implemented using a chemical modulator chip connected to a set of functionalized gold sensing sites downstream. Binding experiments for a model system of carbonic anhydrase-II (CA-II) ligate and immobilized 4-(2-aminoethyl)benzenesulfonamide (ABS) ligand display a one hundred fold (20 dB) improvement in the measured signal-to-noise ratio (SNR) when using the new technique compared to the SNR achieved using the conventional step response method.

The measurement of interactions between biological molecules is of fundamental importance in the life sciences. The most common measured interaction is the binding of two chemical entities. For example one molecule (a ligand) can be immobilized on a surface while exposed to a dissolved analyte binding partner (denoted as ligate). Detection of binding is most commonly achieved by labeling either entity with radioactive or fluorescent markers. Label-free optical detection methods have become increasingly popular within the last decade because they avoid disturbances from conjugated markers or handling of radioactive materials. In particular, label-free optical techniques such as surface plasmon resonance (SPR) permit the study of biological interactions in real time also yielding quantitative kinetic parameters.

The most common type of SPR measurement is a single association-dissociation step depicted in the sensorgram of FIG. 1C with corresponding association and dissociation constants kₐ and k_{d}.

During the cycle, the ligand-ligate complex obeys the differential equation

$$\frac{d C}{d t} = {k}_{a} \cdot A \cdot ( {B}_{0} - C ) - {k}_{d} \cdot C \qquad ( 1 )$$

where B₀ is the surface concentration of the undisturbed immobilized ligand, A is the ligate concentration and C is the surface concentration of the ligand-ligate complex. For a sudden step change in the ligate concentration of width Tₚ, solutions of Eq. (1) obey the piecewise exponential

$$C ( t ) = ( \frac{{k}_{a} \cdot A \cdot {B}_{0}}{{k}_{a} \cdot A + {k}_{d}} ) \cdot ( I - {e}^{- ( {k}_{a} \cdot A + {k}_{d} ) \cdot t} ) \qquad ( 2 )$$

for 0<t≦Tₚ, and

$$C ( t ) = ( \frac{{k}_{a} \cdot A \cdot {B}_{0}}{{k}_{a} \cdot A + {k}_{d}} ) \cdot ( I - {e}^{- ( {k}_{a} \cdot A + {k}_{d} ) \cdot {T}_{P}} ) \cdot {e}^{- {k}_{d} \cdot ( t - {T}_{P} )} \qquad ( 3 )$$

for Tₚ<t≦T. The SPR sensorgram specified by the imager intensity I(t) is proportional to the bound ligate-ligand complex C(t) by the factor G_{SPR}.

$$\begin{matrix}I ( t ) = {G}_{SPR} \cdot C ( t ) \\ = {G}_{SPR} \cdot ( \frac{{k}_{a} \cdot A \cdot {B}_{0}}{{k}_{a} \cdot A + {k}_{d}} ) \cdot ( 1 - {e}^{- ( {k}_{a} \cdot A + {k}_{d} ) \cdot {T}_{P}} ) \cdot {e}^{- {k}_{d} \cdot ( t - {T}_{P} )}\end{matrix} \qquad ( 4 )$$

Eq. (4) represents the idealized SPR sensorgram response. Typically SPR sensors are also sensitive to the bulk characteristics of the solvent which vary slightly in the association and dissociation steps thus producing a bulk output s(t). In addition, the sensorgram signal is subject to drifts and noise originating from a variety of environmental sources (temperature, etc) and the imaging equipment. These effects can be lumped together as disturbance d(t) added to Eq. (4).

$$I ( t ) \approx {G}_{SPR} \cdot ( \frac{{k}_{a} \cdot A \cdot {B}_{0}}{{k}_{a} \cdot A + {k}_{d}} ) \cdot ( I - {e}^{- ( {k}_{a} \cdot A + {k}_{d} ) \cdot {T}_{P}} ) \cdot {e}^{- {k}_{d} \cdot ( t - {T}_{P} )} + s ( t ) + d ( t ) \qquad ( 5 )$$

The solvent bulk contribution s(t) can largely be subtracted out of Eq. (5) using independent measurements of s_{ref}(t) from a non-reacting reference surface, but the disturbance d(t) generally cannot be completely eliminated by referencing as G_{SPR} is disturbance dependent.

In a typical SPR experiment the sensor signal is recorded as a function of time. After the bulk solvent contribution is subtracted out, the remaining signal is fitted to the idealized exponentials of Eq. (2) to determine the kinetic constants. The geometrical interpretation of the step response fitting procedure using conventional methods such as the Levenberg-Marquardt technique requires orthogonality between the Jacobian of the exponential and the disturbance d(t). This poses a problem because the exponential pulse is not orthogonal to common non-oscillatory disturbances such as slow drifts. Therefore in the presence of substantial noise and systematic drifts, the step response fitting scheme is only able to accurately detect relatively coarse interactions between ligate and ligand. In drug discovery applications this is a major limitation, as often drug targets are large proteins with masses of 10⁴-10⁵ Da or larger, and the interaction of interest is often triggered by the binding of a very small ligate (300 Da or less). In order to increase the sensitivity of these systems, commercial state of the art instruments such as the Biacore T100 use many corrective schemes including sophisticated referencing, data scrubber software, temperature control, and most successfully the use of high-density binding sites on three dimensional dextran surfaces (as the SPR sensor detect changes roughly within 100 nm off the surface). In spite of the commercial availability of Biacore systems for many years, these systems have not been able to sense interactions of small ligates, presently 100 Da being the minimum detectable mass. One option for further improvements in detection limits of SPR based systems lies in the development of more robust sensing techniques that reject experimental noise and disturbances more efficiently.

Frequency Domain Signal-Locking Scheme

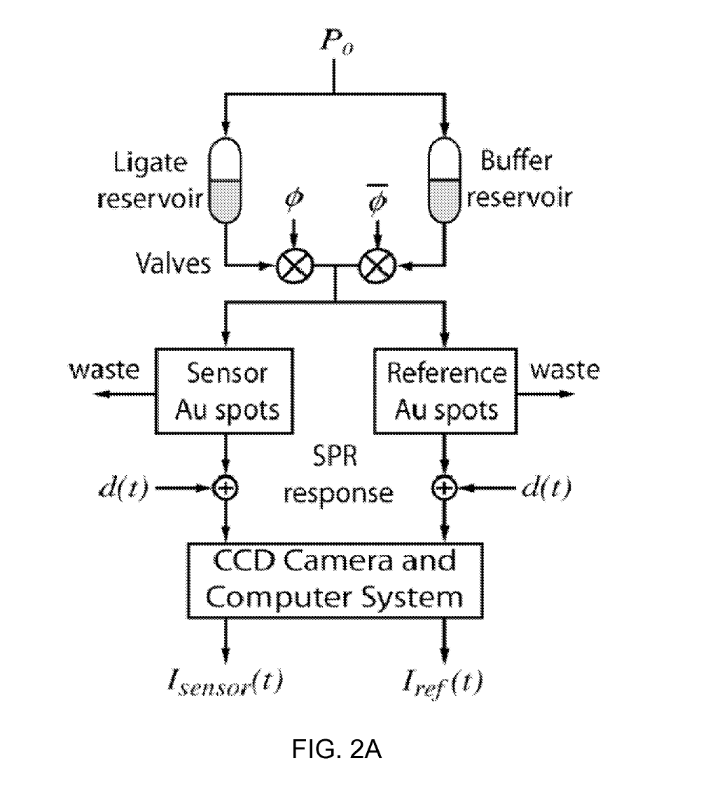

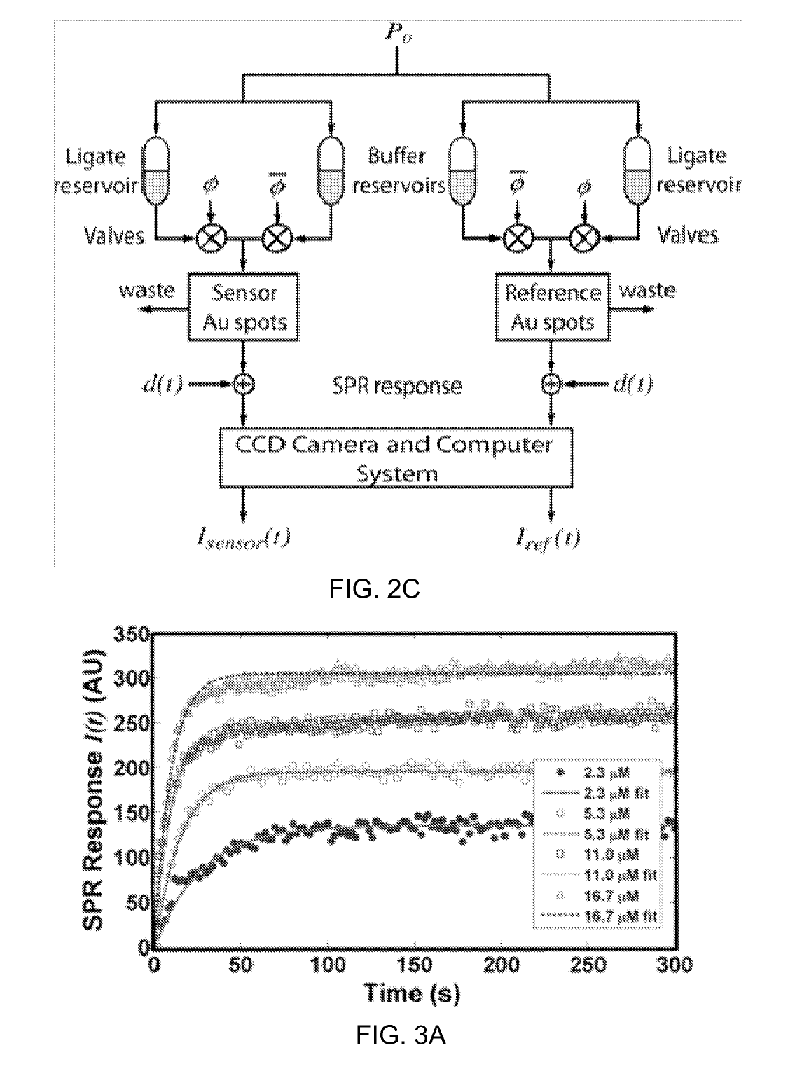

The step response detection method discussed above is limited because it does not attempt to reject the influence of the disturbance on the fit. If the biochemical interaction is linear, it is possible to substantially improve the sensitivity of the measurement by appropriate selection of the input test signal. In particular, an improved detection scheme is implemented if the ligate excitation and the corresponding interaction response are highly correlated. When the ligate input signal and the corresponding sensorgram response have sharp autocorrelations, the input signal and the response are nearly orthogonal to all other signals except themselves; hence rejecting greatly the influence of uncorrelated noise and disturbances. An example of such an approach is the Fourier transform SPR (FT-SPR) detection scheme shown in the diagram of FIG. 2A. In this method the biochemical system is driven by a periodic input excitation of ligate or buffer of constant frequency f_{φ} specified by digital clocks φ and φ̅. FIG. 2C shows an alternate arrangement that utilizes two modulators instead of one. This alternate arrangement avoids problems associated with non-uniform analyte distributions along the channel width which can occur due to the planar layout of the chip modulator if there is insufficient distance between the modulator exit and the sensing site to produce sufficient mixing within a single ligate plug. In one aspect, the channel can be short enough to avoid loss of pulsed analyte concentration but long enough to allow mixing within each analyte/ligate plug.

The corresponding periodic association and dissociation cycles produce a highly correlated sensorgram output of the same frequency, and at steady state of constant amplitude. This detected signal can be locked with respect to the driving clock. The periodic sensorgram thus can be easily detected even in the presence of high levels of additive uncorrelated disturbances and noise. The virtues of the scheme are visibly evident when the power spectrum of the SPR response and the disturbance are plotted as shown in the example of FIG. 2B. While noise and disturbance have broad spectra, the power of the sensorgram dissociation and association response cycles are concentrated at a single modulation frequency thus showing as a spike in the spectrum that can be easily discriminated from the noise using a narrow band filter. Most of the noise and disturbance power falls outside this narrow band; hence it is greatly rejected. The improvement in the noise rejection will be quantitatively analyzed in the following section.

Small-Signal Biochemical Transfer Function

In order to utilize these signals effectively a relation between the excitation response and the kinetic constants can be established. The clock frequency f_{φ} can be set and record the sensorgram response. By repeating this procedure at different discrete modulation frequencies one may obtain a relation between the excitation frequency and the sensorgram response. Since both excitation and response are periodic, they can be expanded as Fourier series. The relation between the fundamental terms of response and input series is the Fourier transfer function (TF) H(jω) of the biochemical system

$$H ( jω ) = \frac{C ( jω )}{A ( jω )} = \frac{{c}^{⋒}}{\hat{a}} = \frac{{k}_{a} \cdot {B}_{o}}{( {k}_{d} + {k}_{a} \cdot {A}_{o} )} \cdot \frac{1}{( 1 + jω / p )} \qquad ( 6 )$$

where C(jω) and A(jω) are complex phasors of the input ligate excitation and filtered sensorgram output respectively, pole p=k_{d}+kₐA₀, and ω=2πf_{φ} is the excitation angular frequency. For small periodic input perturbations of the type

$$A ( t ) = {A}_{0} + Δ A \cdot ( t ) = {A}_{0} + {\sum }_{n = 1}^{∞} Δ {A}_{n} sin ( n \cdot ω \cdot t ) \qquad ( 7 )$$

with |ΔA(t)|≦0.1·A₀, the sensorgram response is proportional to the ligate-ligand complex which also has the form

$$C ( t ) = {C}_{0} + Δ C \cdot ( t ) = {C}_{0} + {\sum }_{n = 1}^{∞} {c}_{n} sin ( n \cdot ω \cdot t + {θ}_{n} ) , \qquad ( 8 )$$

where C₀ is the equilibrium ligate-ligand concentration. If the biochemical interaction is determined by Eq. (1), the transfer function is directly related to the kinetic constants kₐ and k_{d}, and it is obtained by direct substitution of Eqs. (7) and (8) into Eq. (1)

$$\frac{d Δ C ( t )}{d t} = {k}_{a} \cdot ( {A}_{0} + Δ A ( t ) ) \cdot ( {B}_{0} - {C}_{0} - Δ C ( t ) ) - {k}_{d} \cdot ( {C}_{0} - Δ C ( t ) ) . \qquad ( 9 )$$

After some algebra this reduces to

$$\begin{matrix}\frac{d Δ C ( t )}{d t} = {k}_{a} \cdot ( {B}_{0} - Δ C ( t ) ) \cdot Δ A ( t ) - ( {k}_{a} \cdot {A}_{0} + {k}_{d} ) \cdot Δ C ( t ) \\ \approx {k}_{a} \cdot {B}_{0} \cdot Δ A ( t ) - ( {k}_{a} \cdot {A}_{0} + {k}_{d} ) \cdot Δ C ( t )\end{matrix} \qquad ( 10 )$$

Eq. (10) is valid for correspondingly small changes in the ligate-ligand complex such that B₀>>ΔC(t). The reduced simplified equation is linear in ΔA(t) and its corresponding Fourier series components. The small-signal transfer function H(jω) is easily obtained from Eq. (10) by substitution of the time derivative with jω. The small-signal sensorgram transfer function at the fundamental component has the single pole behavior

$$\begin{matrix}H ( jω ) = {G}_{SPR} ( \frac{{c}_{1}}{Δ {A}_{1}} ) {e}^{j {θ}_{1}} \\ = {G}_{SPR} \frac{{k}_{a} \cdot {B}_{0}}{( {k}_{a} \cdot {A}_{0} + {k}_{d} )} \cdot \frac{1}{( 1 + jω / {ω}_{p} )} ,\end{matrix} \qquad ( 11 )$$

with characteristic pole ωₚ

ωₚ=2πfₚ=kₐ·A₀+k_{d}. (12)

Therefore, the kinetic constants kₐ and k_{d} can be experimentally determined by direct measurements of the transfer function and its characteristic pole for different A₀ ligate concentrations.

Sensorgram Under Large Signal Excitation.

In the FT-SPR measurement it is advantageous to increase the excursion of the input ligate beyond 0.10A₀ because the magnitude of the corresponding sensorgram increases proportionally. For large ligate excursions the analysis is more complex because Eq. (1) differs for the association and dissociation cycles and input-output linearity of the Fourier components does not hold. In the large signal analysis, the ligate excitation can be assumed a periodic square waveform of period T, amplitude A₀ and clock frequency f_{φ}=1/T. At low frequencies the sensorgram is also square, but as the frequency increases it becomes triangular thus changing the Fourier coefficients of the response. The impact of the change in shape can be accounted by calculating the Fourier series components of the sensorgram waveform. At steady state, C(0)=C(T); hence the characteristic solutions of Eqs. (2) and (3) can be set equal to each other

a−b=(a−b·e^{-β}^{₁})·e^{-β}^{₂}, (13)

with

$$a = ( \frac{{k}_{a} \cdot A \cdot {B}_{0}}{{α}_{1}} ) , {α}_{1} = {k}_{a} \cdot A + {k}_{d} , {α}_{2} = {k}_{d} , {β}_{1} = \frac{{α}_{1} \cdot T}{2}$$

and

$${β}_{2} = \frac{{α}_{2} \cdot T}{2} .$$

Solving Eq. (13) for b, the steady state solution can be expressed as

$$C ( t ) = \{ \begin{matrix}a ( 1 - [ \frac{1 - {e}^{- {β}_{1}}}{1 - {e}^{- ( {β}_{1} + {β}_{2}} )} ] \cdot {e}^{- {α}_{1} \cdot t} ) , & 0 \leq t \leq \frac{T}{2} \\ a ( 1 - [ \frac{( 1 - {e}^{- {β}_{1}} ) \cdot {e}^{- {β}_{1}}}{1 - {e}^{- ( {β}_{1} + {β}_{2}} )} ] ) \cdot {e}^{- {α}_{2} \cdot ( t - \frac{T}{2} )} , & \frac{T}{2} \leq t \leq T .\end{matrix} \qquad ( 14 )$$

The first component of the complex Fourier series of Eq. (14) can be determined for two limiting conditions as T→∞(ω→0) and T→0 (ω→0) while replacing the exponentials with simplified first term expansions. When T→∞ the exponential solution becomes a plain square wave of frequency f_{φ} with corresponding first (or fundamental) complex Fourier series coefficients

$${C}_{1}^{∞} = \frac{1}{T} {\int }_{0}^{T / 2} a \cdot {e}^{- j \frac{2 π \cdot t}{t}} d T = - j \frac{a}{π} , \\ {C}_{- 1}^{∞} = \frac{1}{T} {\int }_{0}^{T / 2} a \cdot {e}^{+ j \frac{2 π \cdot t}{T}} d t = + j \frac{a}{π} \qquad ( 15 )$$

The first component of the complex Fourier series is

$$\begin{matrix}{c}_{1}^{∞} ( t ) = - \frac{j a}{π} {e}^{j \frac{2 π t}{T}} + \frac{j a}{π} {e}^{- j \frac{2 π t}{T}} \\ = \frac{2 a}{π} sin ( \frac{2 π t}{T} ) \\ = {c}_{1}^{∞} \cdot sin ( \frac{2 π t}{T} ) .\end{matrix} \qquad ( 16 )$$

The amplitude c₁^{∞}=2a/π of the sinusoidal component will be used later on to find the magnitude of the normalized transfer function. For the case when T→0, the complex Fourier series coefficients at the fundamental are

$$\begin{matrix}{C}_{1}^{0} = \frac{1}{T} {\int }_{0}^{T / 2} a ( 1 - [ \frac{1 - {e}^{- {β}_{1}}}{1 - {e}^{- ( {β}_{1} + {β}_{2} )}} ] \cdot {e}^{- {α}_{1} \cdot t} ) \cdot {e}^{- j \frac{2 π t}{T}} d t + \frac{1}{T} {\int }_{T / 2}^{T} a ( 1 - [ \frac{( 1 - {e}^{- {β}_{1}} ) \cdot {e}^{- {β}_{1}}}{1 - {e}^{- ( {β}_{1} + {β}_{2} )}} ] ) \cdot {e}^{- {α}_{2} ( t - \frac{T}{2} )} \cdot {e}^{- j \frac{2 π t}{T}} d t , & ( 17 ) \\ {C}_{- 1}^{0} = \frac{1}{T} {\int }_{0}^{T / 2} a ( 1 - [ \frac{1 - {e}^{- {β}_{1}}}{1 - {e}^{- ( {β}_{1} + {β}_{2} )}} ] \cdot {e}^{- {α}_{1} \cdot t} ) \cdot {e}^{+ j \frac{2 π t}{T}} d t + \frac{1}{T} {\int }_{T / 2}^{T} a ( 1 - [ \frac{( 1 - {e}^{- {β}_{1}} ) \cdot {e}^{- {β}_{1}}}{1 - {e}^{- ( {β}_{1} + {β}_{2} )}} ] ) \cdot {e}^{- {α}_{2} \cdot ( t - \frac{T}{2} )} \cdot {e}^{+ j \frac{ 2 π t}{T}} d t . & ( 18 )\end{matrix}$$

Taking the limit of Eqs. (17) and (18) as T→0,

$${C}_{- 1}^{0} = {C}_{1}^{0} = - \frac{{α}_{1} {α}_{2} aT}{( {α}_{1} + {α}_{2} ) {π}^{2}} , \qquad ( 19 )$$

and the fundamental component of the complex Fourier series is

$$\begin{matrix}{c}_{1}^{0} ( t ) = - \frac{{α}_{1} {α}_{2} aT}{( {α}_{1} + {α}_{2} ) {π}^{2 }} \cdot {e}^{j \frac{2 π t}{T}} - \frac{{α}_{1} {α}_{2} aT}{( {α}_{1} + {α}_{2} ) {π}^{2}} \cdot {e}^{- j \frac{ 2 π t}{T}} \\ = - \frac{2 {α}_{1} {α}_{2} aT}{( {α}_{1} + {α}_{2} ) {π}^{2}} cos ( \frac{2 π t}{T} ) \\ = {c}_{1}^{0} cos ( \frac{2 π t}{T} ) .\end{matrix} \qquad ( 20 )$$

The cosine amplitude at the fundamental is

$${c}_{1}^{0} = - \frac{2 {α}_{1} {α}_{2} aT}{( {α}_{1} + {α}_{2} ) {π}^{2} } . \qquad ( 21 )$$

With the amplitude from these two limiting cases an approximation for the magnitude of the normalized large signal transfer function is obtained as

$$\begin{matrix}‖ T ( j ω ) \vert = ‖ \frac{H ( j ω )}{H ( 0 )} ‖ \approx ‖ \frac{{c}_{1}^{0} ( j ω )}{{c}_{1}^{∞}} ‖ \\ = \frac{2 {α}_{1} {α}_{2} aT}{( {α}_{1} + {α}_{2} ) {π}^{2}} \cdot \frac{π}{2 a} \\ = \frac{{α}_{1} {α}_{2}}{( {α}_{1} + {α}_{2} ) π \cdot f} \\ = \frac{2 {α}_{1} {α}_{2}}{( {α}_{1} + {α}_{2} ) ω} .\end{matrix} \qquad ( 22 )$$

At the pole frequency f=fₚ, the magnitude of the normalized transfer function equals 1/√2; hence the expression for the pole for large signal excitation is approximately

$${f}_{p} \approx \frac{\sqrt{2} \cdot {α}_{1} {α}_{2}}{π ( {α}_{1} + {α}_{2} )} . \qquad ( 23 )$$

This can be written finally in terms of kₐ and k_{d} as

$${f}_{p} \approx \frac{\sqrt{2} \cdot ( {k}_{a} \cdot {A}_{0} + {k}_{d} ) \cdot {k}_{d}}{π ( {k}_{a} \cdot {A}_{0} + 2 {k}_{d} )} . \qquad ( 24 )$$

Equation (24) provides a relation between the observed pole and the kinetic constants under the condition of large ligate square wave excitation. Note that this expression differs significantly from the small signal pole expression of Eq. (12). In particular Eq. (24) includes the effects of saturation which develop for large A₀ making the pole approach √2·k_{d}/π.

SPR System Implementation.

The FT-SPR technique involves sweeping of the ligate modulation frequency and the generation of a corresponding stream of ligate plugs through to the SPR sensing surface. In general one may generate such plug stream using a set of valves that periodically switch equal flows of buffer and ligate solutions through the SPR sensing sites. This methodology works well for slow switching plugs, but in order to create short fast plugs, microfluidic modulator chips can be used that permit the transport of time-dependent chemical signals with relatively low dispersion. One example of such a system is discussed below.

### Example 1

An amount of 5 KDa carboxymethyl-PEG-thiol (CM-PEG) and 2 KDa methoxy-PEG-thiol (M-PEG) were purchased from Laysan Bio. Sulfo-Nhydroxysuccinimide (S-NHS), N-ethyl-N′-(3-dimethylaminopropyl)carbodiimide (EDC), and phosphate buffered saline (PBS) tablets were obtained from Thermo Scientific. The 4-(2-Aminoethyl)benzenesulfonamide (ABS), carbonic anhydrase II (CA-II), and 10% sodium dodecyl sulfate (SDS) solution were from Sigma. Sylgard 184 from Dow Chemicals was used to make the PDMS. SF10 glass substrates in a 2″×2″ square were purchased from Schott glass. An 18 MΩ water was used for rinsing and all buffers used was provided by an in-house plant associated with the University of Utah Nanofab.

A GWC Technologies SPRimager®2 system was used to collect the data. The system was modified by the manufacturer to be used in a horizontal configuration, with a custom built microfluidic mounting cell. The instrument was further modified with a World Star Tech TEGCIRL-100G-808 808 nm laser source, a rotating diffuser from Suss-Microoptics, a Navitar Zoom 6000 lens system, and a Hamamatsu C9100-01 EMCCD camera.

The micro fluidic chip was fabricated using a conventional PDMS process on glass substrates. A two-layer channel system was used to create valves to enable switching between buffer and ligate. The PDMS was molded over photolithographically defined patterns using SU-8 and AZ9260 photoresists. The two layers were cleaned and exposed to oxygen plasma for 12 seconds at a pressure of 400 mTorr in a March Plasmod system, and then aligned and bonded together using an in-house built alignment system. The system contained two sets of channels, one to act as a reference (control) and the other for detection of the ligate of interest (sensor). The channels are 180 μm wide and 23 μm tall, with the sensing spots starting at a distance of 2 mm downstream from the valves.

The glass substrate was cleaned by piranha etch (3:1, H₂SO₄:H₂O₂) for 20 minutes, rinsed for 5 min in 18 MΩ deionized (DI) water, and dried under N₂. The substrates were placed in an oven at 80° C. for 10 minutes before being transferred to a TMV sputter deposition system. A 3 nm adhesion layer of Ti/W was deposited, followed by 40 nm of gold. The metal layers on the substrate were patterned using photolithography to make an array of 140×180 μm SPR sensing sites. The metals were etched and the samples rinsed thoroughly with 18 MΩ DI water after each etch step to prevent contamination. Finally, the photoresist was removed and the patterned substrates stored in a desiccator until used.

Before assembly with the PDMS microfluidic channels, the patterned glass substrates were further cleaned by flame annealing with a butane microtorch. This was followed by ultrasonication in methanol for five minutes. The methanol was refreshed and the substrate sonicated a total of three times. The dual layer PDMS channels were drilled to make through holes for fluid access, and then cleaned with acetone, IPA, and DI water. The PDMS was dried with an air gun and then baked for 10 minutes on a hot plate at 100° C., followed by a cooling step for 10 minutes. At this point, both the glass substrate and PDMS channels were placed in the oxygen plasma cleaner for 12 seconds at the same settings mentioned previously. Alignment was performed to coordinate the channels with the patterned gold surface, and then the assembled chip was baked, again for 10 minutes at 100° C.

Modification of the gold spots was performed in situ to enable the bonding of the PDMS and glass substrate. The assembled chip was connected to a syringe pump and flushed with 0.01 M HCl for 5 minutes to remove any contaminants or oxide, followed by a 15 minute rinse with DI water. A mixture of long and short chain polyethylene glycol (PEG) molecules was used to provide both a capture surface and one that is protein resistant, following the protocol described by Uchida et al, Anal. Chem., 2005, 77, pp. 1075-1080 which is incorporated herein by reference. The CM-PEG molecule was introduced to the flow channels for 20 minutes, followed by a 0.05 M NaOH rinse. Then the M-PEG was passed through the channels and rinsed similarly. The smaller PEG was applied a total of three times to backfill any gaps in the initial PEG layer and prevent protein adsorption on the gold surface. The surface was again treated with 0.01 M HCl and rinsed with DI water to ensure the proper chemical functionality for the next step.

Chemical modification of the CM-PEG chains was performed using S-NHS and EDC to create an amine reactive surface. S-NHS at 100 mM and EDC at 400 mM in water are mixed 1:1 and passed through both the reference and sensing channels for 30 minutes. The system was rinsed with PBS for two minutes, and then the capture molecule applied. ABS was passed over the sensing surface, while only the buffer was passed over the reference surface, illustrating the need for the dual channel system. Finally, both surfaces were blocked with ethanolamine in carbonate buffer at pH 9 for 30 minutes and rinsed with PBS.

The microfluidic chip was placed in the SPR mounting cell, with n=1.72 refractive index matching fluid to couple between the prism and glass substrate. Microfluidic connections were made to the valves and flow channels, and then the valves control lines were filled with water, due to the gas permeability of PDMS. The valves were actuated by a pressure system at 30 psi controlled by a computer. Syringes containing ligate (CA-II), buffer (PBS), and regeneration solution (0.1% SDS in PBS) were pressurized as well with a constant source at 10 PSI which corresponds to a flow rate of 16 cm/s, sufficiently high to overcome transport limiting effects. Four different concentrations of CA-II were used, 2.3, 5.3, 11.0, and 16.7 μM. Experiments were performed for each CA-II concentration by collecting the response from a single step input first, and then continuously running multiple cycles of a square wave input at different frequencies, from 2 mHz to 250 mHz. The sampling rate was 4 Hz for all experiments. Wasabi camera control software from Hamamatsu was used to analyze the collected data by selecting multiple sensing and reference spots in the image. The mean intensity value in each spot I(t) was recorded for all of the frames in the experiment. Fourier transform analysis of the data was performed in Matlab. After an experiment at a given concentration was completed, the channels were flushed with regeneration solution to clean the sensor surface.

Results and Discussion

Kinetic constants were obtained using both a single step method and the large-signal FT-SPR scheme. In all the data sets the fixed imager intensity offset I_{offset} was first recorded at the beginning of the measurement and subtracted from all the recorded data. Each spot analyzed comprised approximately 1550 pixels on the CCD camera.

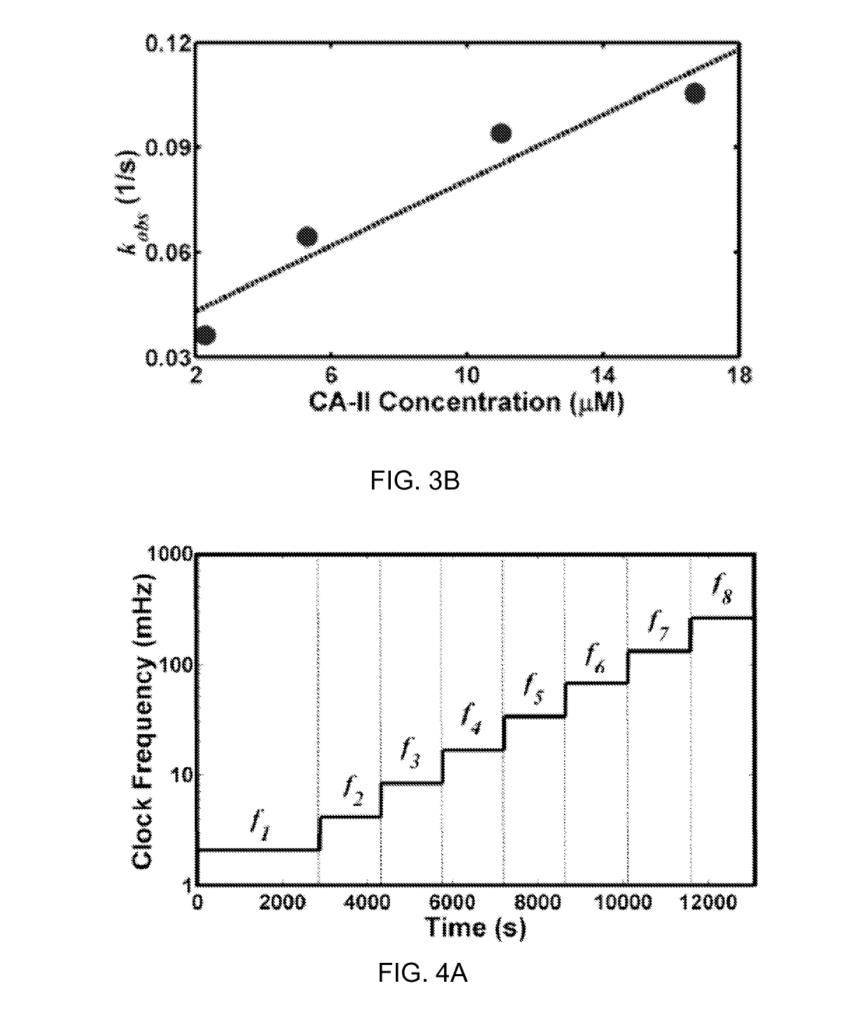

For each concentration a single step experiment was first performed by flowing buffer for 5 minutes, CA-II for 5 minutes, and then buffer again for 5 minutes. After removing I_{offset} from the data, the signal from a reference spot, I_{ref}, was subtracted from a sensor spot, Iₛₑₙₛₒᵣ, at the same distance downstream from the fluid control valves. This is needed to cancel the solvent bulk contribution s(t) of the ligate solution as compared to that of the buffer. The corrected responses from the single step experiments are shown in FIG. 3A, along with the fits to the exponential solution

I(t)=I₀(1−e^{-k}^{_{obs}}^{·t}). (25)

where k_{obs} is the observed rate constant for the association phase,

k_{obs}=kₐ·A₀+k_{d}. (26)

The observed rate values were then plotted against the respective concentration values and a linear fit was used to determine kₐ and k_{d} according to Eq. (26) as shown in FIG. 3B. These values were found to be 4.7×10³ l/M·s and 3.4×10⁻² l/s respectively in reasonable agreement with published results.

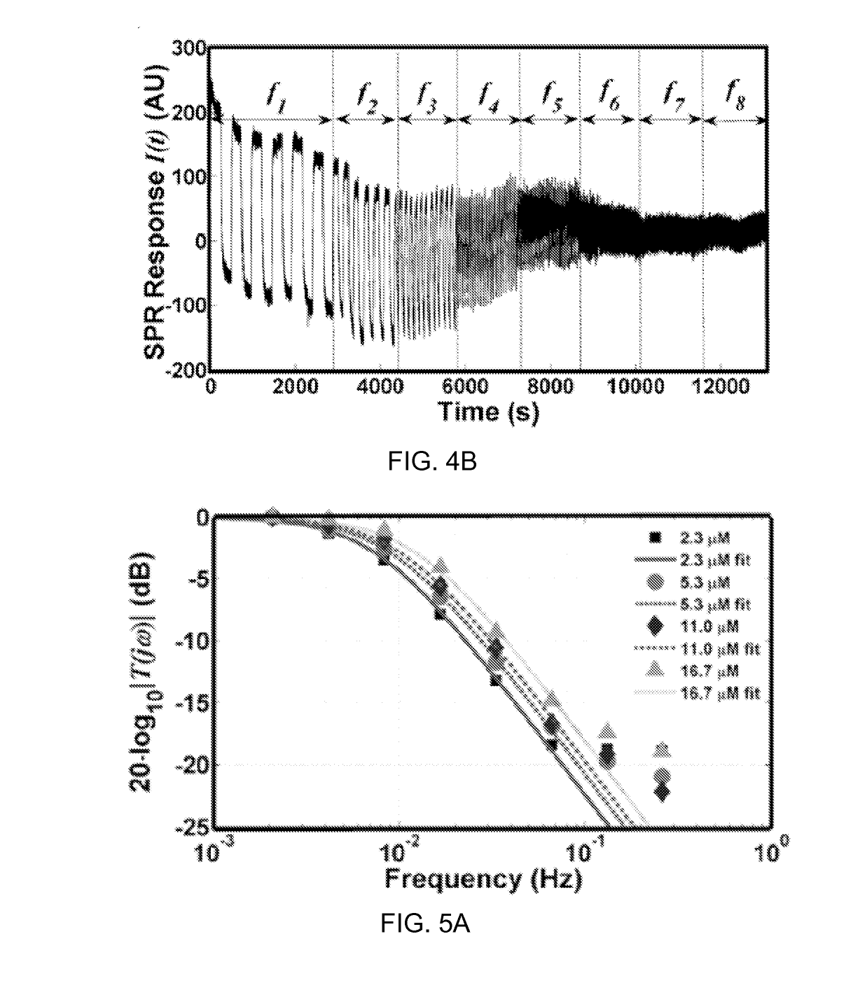

The continuous cycle intensity data was collected for each concentration at a discrete set of frequencies selected to give an even power distribution across the spectrum. The input frequencies and output waveforms are shown in FIG. 4A and FIG. 4B, respectively. The frequencies began at 2 mHz and increased by doubling until 250 mHz was reached, keeping the time each frequency was applied constant. This was performed after the single step data was collected so that the system was near steady state when the cycles started to avoid a long transition during the experiment.

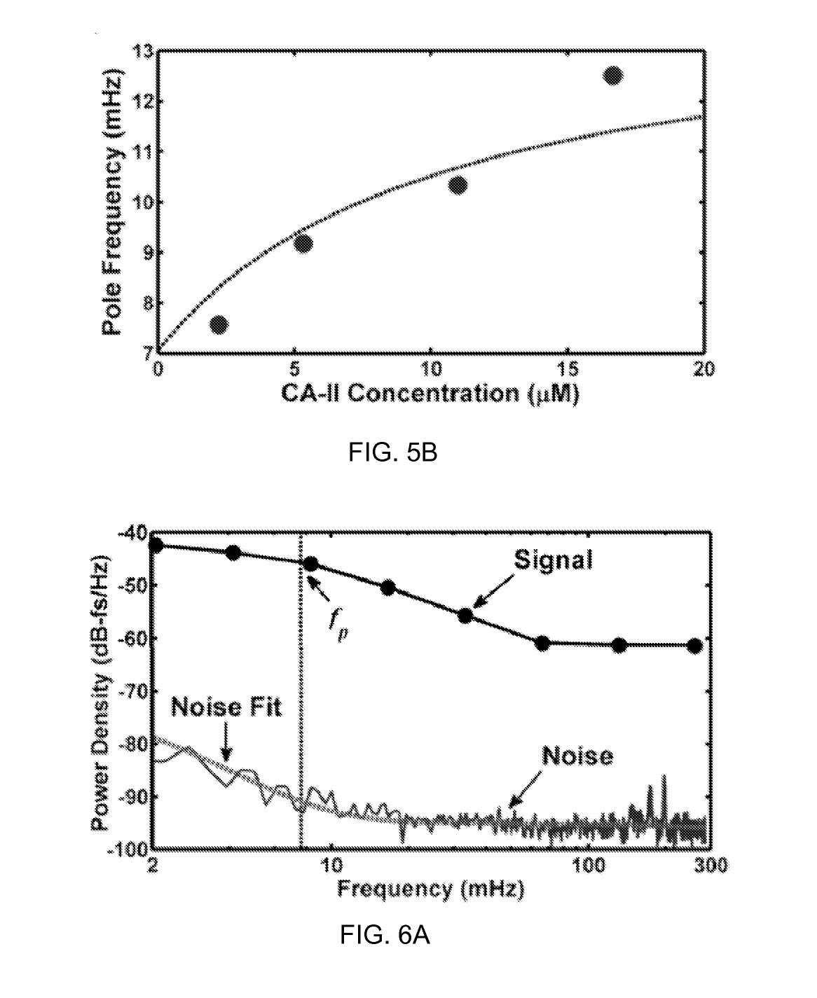

Fourier transform analysis is used to determine the magnitude of the sensorgram response intensity and normalized transfer function ∥T(jω)∥ at the excitation frequencies from the magnitude of the measured power spectrum. ∥T(jω)∥ was next plotted versus each excitation frequency as shown in the Bode plot of FIG. 5A. The pole frequency for each concentration was determined as the 3 dB point according to

$$‖ T ( j ω ) ‖ = \frac{1}{\sqrt{1 + {( f / {f}_{p} )}^{2}}} . \qquad ( 27 )$$

The kₐ and k_{d} determined from the FT-SPR analysis by fitting the pole values to Eq. (24) as shown in FIG. 5B. They were found to be 6.1×10³ l/M·s and 3.2×10⁻² l/s respectively. These values match well with the single step determined values reported above. The values determined by the FT-SPR method do differ slightly from reported values of kₐ and k_{d} for the same model system, but the range of these values is very large among the different detection methods used. The difference in the values obtained can be attributed to the novel surface chemistry employed in this example.

The signal to noise ratio (SNR) in decibels (dB) for the step response measurement was computed from the ratio of the average power of the exponential fitted signal to the average power of the fit residual over the measurement period as shown below

$${SNR}_{step} = 10 log ( \frac{{\int }_{0}^{τ} {I}_{fit}^{2} d t}{{\int }_{0}^{τ} {( {I}_{meas} - {I}_{fit} )}^{2} d t} ) . \qquad ( 28 )$$

The measured SNR for the step response method was 26 dB meaning that the average power of the fitted exponential was 400 times larger than that of the noise and disturbances.

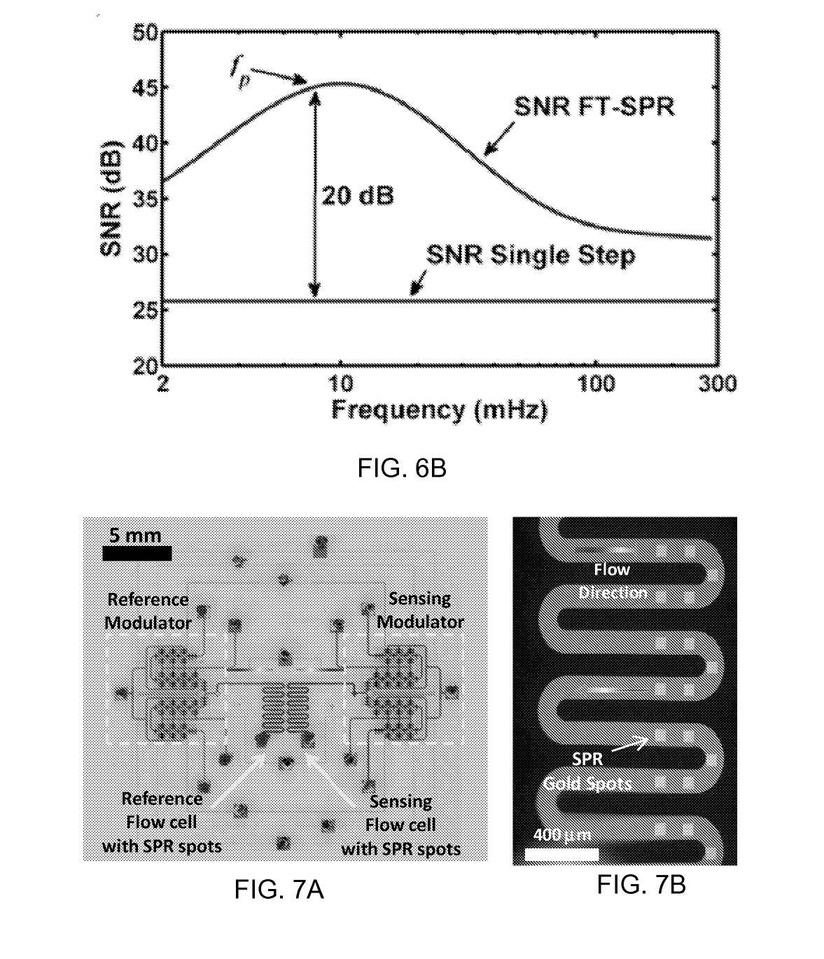

The SNR for the FT measurement was calculated differently from that of the step response. First, the noise and disturbance intensity signal of the SPR system under no input excitation was recorded for 30 minutes at a sampling frequency of 4 Hz. After subtraction of the initial offset, the intensity was normalized to the full scale of the imaging camera. The power spectral density of the normalized intensity signal measured in dB-full scale/Hz (or dB-fs/Hz) was then computed. Because this is a random signal, the power spectrum was averaged over 10 equal duration 180 second recording cycles. This procedure produces a reasonably well defined estimate of the disturbance and noise spectral density. The relative power of the system noise and disturbances to the power spectrum of the FT-SPR signal (also in dB-fs/Hz) can be compared as shown in FIG. 6A. Note that the power spectrum of the FT-SPR interaction signal is simply ∥H(jω)∥² which is flat at low frequencies and displays a dual pole at fₚ. In contrast, the power spectrum of the noise and disturbance seems to be largely concentrated at the lower frequencies becoming flat at the higher frequencies. The SNR for the FT-SPR measurement is equal to the ratio of the two power spectrums as shown in FIG. 6B. Note that unlike the fixed SNR for the step response method the SNR of the FT-SPR technique increases and peaks at a specific frequency near the TF pole. In addition the peak SNR is much higher for the FT-SPR technique than the step response method by a factor of 20 dB or 100-fold better than that of the step response method. At least two reasons appear to be behind the observed SNR improvement; (a) the measurement takes place in a region where the power density of the disturbances and noise is low and (b) the power spectrum of the disturbance is broad hence only a small contribution of the disturbance and noise is present at the narrow excitation frequency. The major improvement in the SNR of the narrowband FT-SPR technique indicates that direct detection of small molecule binding on these surfaces can be achieved.

Finally, in spite of the impressive SNR ratios, the FT-SPR technique does have practical limitations. First, in order to make a clean measurement, the modulator chip can produce sufficiently high frequency plugs to reach the TF pole region. Therefore, small distances between the chip valves and the sensing spots are recommended to minimize the plug dispersion. In addition, smaller noisier spots can be used in the chip. The technique also involves periodic excitation and a periodic response, therefore it cannot handle irreversible reactions with strong associations and weak dissociation, but such reactions could be measured with the use of periodic regeneration cycles.

This example illustrates an implementation of a signal locking Fourier transform SPR scheme for the detection of biomolecular interactions under periodic ligate excitation. Using this method kinetic values of kₐ and k_{d} were obtained for a CA-II binding model experiment similar to those obtained from SPR data analysis on the given surface, with the single step determined kₐ=4.7×10³ l/M·s vs. the FT value of 6.1×10³ l/M·s, and similarly k_{d}=3.4×10⁻² l/s vs. 3.2×10⁻² l/s. When compared to the step response methodology, the FT-SPR method produces 100-fold improvements in the SNR of the measured signal due to the narrowband nature of the technique which rejects very well the uncorrelated noise and disturbances.

The microfluidic chip assembly method used prevents multiplexing on the sensor surface as the approach can only look for one thing at a time. However, altering the assembly method to attach the microfluidics after the surface is functionalized can provide the ability to multiplex. For example, sensor spots can be functionalized with different targets or the sensor system can be replicated on a larger substrate (e.g. for various drugs). In another aspect, a common sensor can be used to multiplex multiple compounds by exposing multiple compounds in a common flow. The repeating pattern can produce spatially-separated compounds which can be analyzed independent of one another (e.g. via segmented flow with sections or plugs of flow with different drugs). Also, there may be limitations to the channel length, and therefore the number of spots available for detection, to achieve the needed signal profile. This can be overcome by increasing the number of fluid channels. The biosensor can also be used to simultaneously monitor multiple reactions. For example, if there are two substances present in the fluid that react with the functionalized SPR target surface, and if these interactions have corresponding poles of sufficiently different frequencies, then the two binding phenomena (the two poles) would be observable in a single frequency sweep.

### Example 2

### Multisine SPR Biosensor

FIGS. 7A and 7B show photographs of a dual (sensor and reference) channel chip, fabricated using conventional two-level PDMS, and embedded Au sensing surface on the glass substrate. Each channel consists of two interleaved multiplug pulse encoded modulators connected to a flow cell with gold spots. The signal generators produce a periodic stream of protein flowing at constant velocity through the cell. The chosen test signals have a well defined spectrum and are useful to determine spectral measurements at several frequencies simultaneously in a time efficient manner. The analyte excitation scheme chosen was multisine waveforms s(t) of the type

$$s ( t ) = {A}_{avg} + {\sum }_{i = 1}^{N} {A}_{i} cos ( {ω}_{i} t + {θ}_{i} ) \qquad ( 29 )$$

where N is the number of frequency components. Selection of the frequencies such that ωᵢ=k·ω₀, k being an integer, the multisine discrete frequencies are all contained within a single cycle of the lowest component, and the measurement interval corresponds of a few periods of ω₁. The practical generation of a synthetic multisine signal requires careful consideration of component harmonics, their amplitude and relative phases indicated in above equation by θᵢ. The phases are selected to minimize the waveform crest factor

$$C = \frac{{\vert s \vert}_{peak}}{{s}_{r m s}} \qquad ( 30 )$$

which is equal to the peak to root-mean-square average ratio. For a single sinusoid, the crest factor is \sqrt{2}. For a multisine the crest factor is generally higher. A low crest factor is desirable because it avoids the presence of exceedingly large peaks in the waveform and reduces the resolution requirements for waveform generation. A suitable low crest factor multisine is the Schroeder multisine with quadratically weighted phases which is used in this example. Although other crest factors can be chosen, the Schroeder scheme is not the only low crest phase configuration, one may also select the phases via numerical minimization of the crest factor.

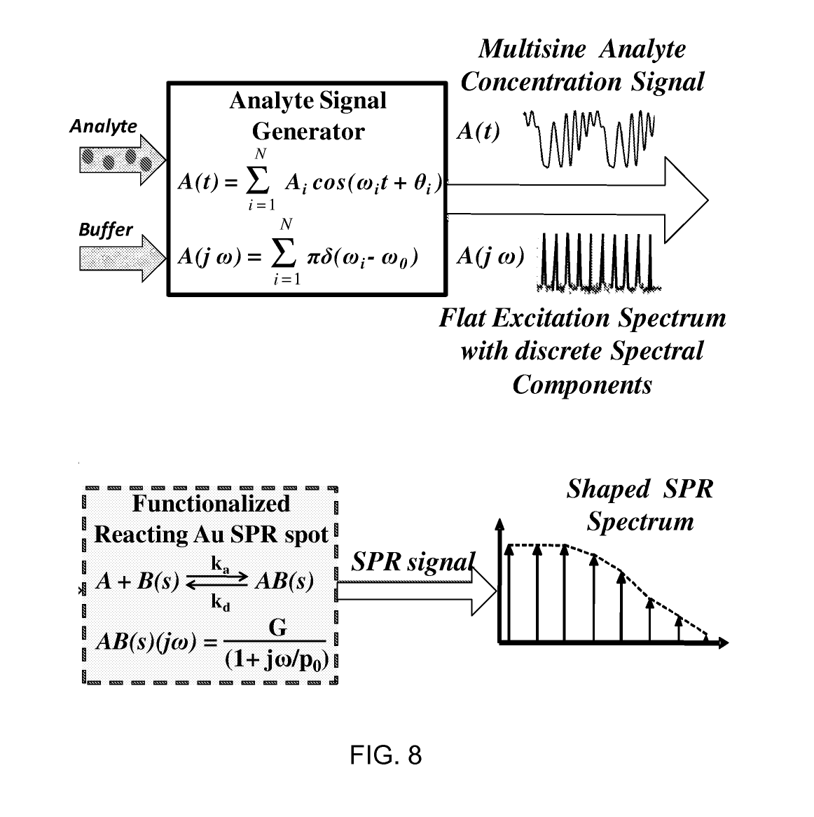

This excitation yields corresponding periodic protein association and dissociation cycles at the functionalized sensing surface. The surface activity was measured optically using SPR imaging. The binding reaction produces poles in the observed frequency response as the excitation (protein) frequency was changed. The pole magnitude is directly related to the association and dissociation constants of the reaction. In the diagram of FIG. 8, the modulators generated an analyte concentration signal including a digitized superposition of sinusoidals (or multisine) with minimum crest factor. The spectrum of the excitation contains a discrete number of frequencies producing corresponding discrete responses. Thus, the spectral content of the signal permits the simultaneous measurement of the transfer function at multiple frequencies. If this input analyte excitation has equally weighed spectral components, after interaction with the functionalized sensing spots the SPR sensorgram will be attenuated for frequencies larger than the pole frequency thus shaping the sensorgram spectrum. The entire measurement can be done in a few cycles of the lowest multisine component thus significantly reducing the measurement time compared to the frequency sweep scheme.

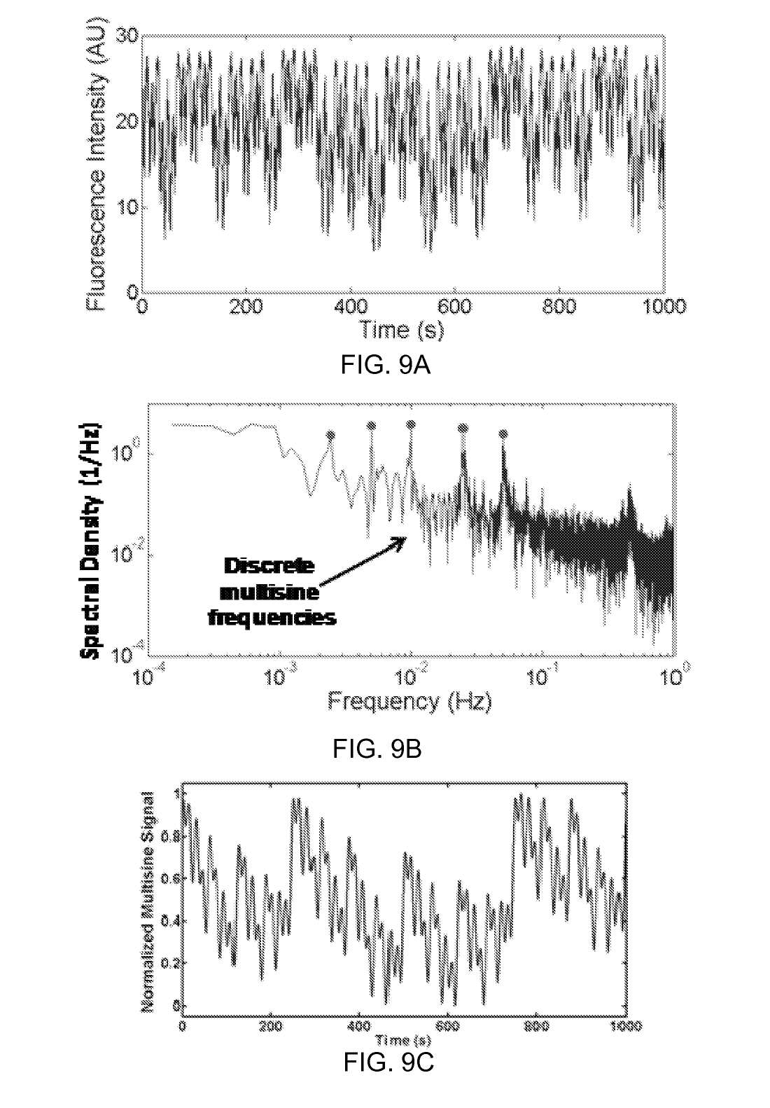

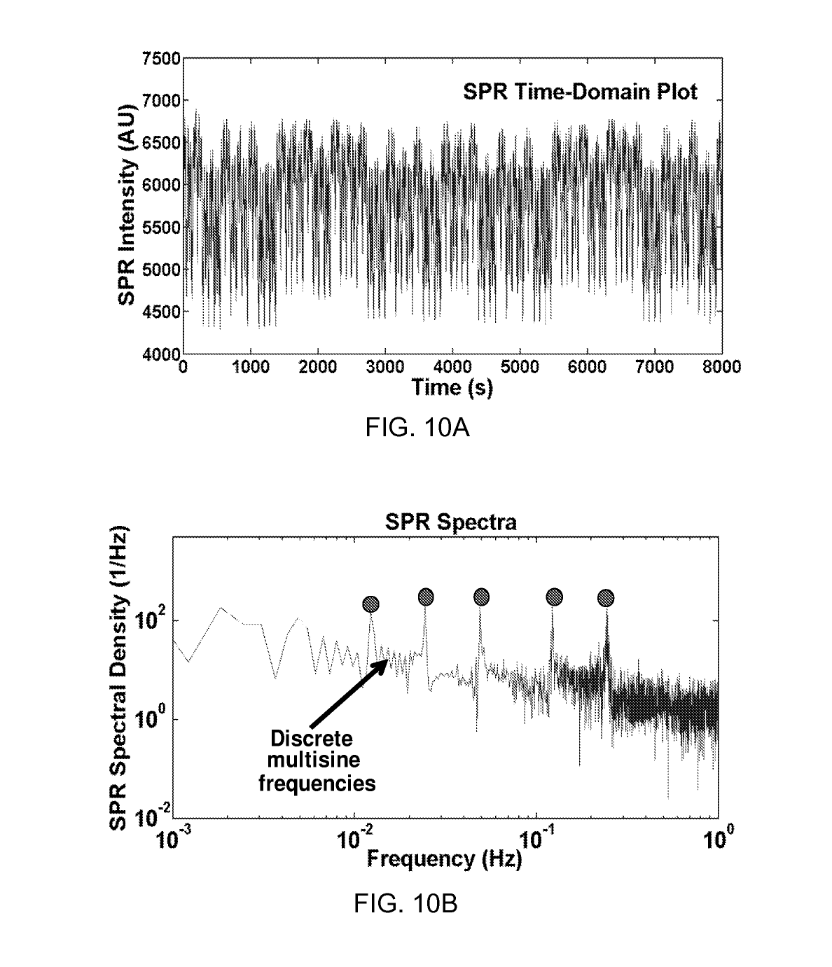

FIGS. 9A and 9B show an example time domain and spectral density response, respectively, of a multisine fluorescent signal from the chip measured with a fluorescent microscope at 2 cm/s flow velocity. FIGS. 10A and 10B show a corresponding example of these plots for a 10% ethanol and water multisine signal measured on the flow cell gold spots using a GWC SPR Imager II system.

Five kDa carboxymethyl-PEG-thiol (cm-PEG) and two kDa methoxy-PEG-thiol (m-PEG) were purchased from Laysan Bio. Sulfo-N-hydroxysuccinimide (S-NHS), N-ethyl-N′-(3-dimethylaminopropyl)carbodiimide (EDC) and phosphate buffered saline (PBS) tablets were purchased from Thermo Scientific. Carbonic anhydrase II protein (CA II), 4-(2-aminoethyl)benzenesulfonamide (ABS) and 10% sodium dodecyl sulfate (SDS) solution were purchased from Sigma. Ethanol (200 Proof) was purchased from Decon Labs, Inc. Sylgard 184 Polydimethylsiloxane (PDMS) kit was purchased from Dow Chemicals and deionized 18 MΩ water (DI water) was used as solvent and for rinsing purposes. A modified GWC Technologies SPRimager2 system adapted to accommodate our chip-on-substrate was employed for extracting sensorgram data.

SF10 glass substrates (2×2 inch) were cleaned by piranha etch for 10 mins followed by DI water rinsing for 5 mins and blow dried with N₂ prior to metal deposition. This was followed by deposition of Ti/W adhesion layer and Au layer. The metal layers are then patterned using photolithography. The patterned array of SPR sensing gold spots have dimensions of 125×125 μm² and thickness of ˜45 nm. The metals were then etched, the photoresist was removed and finally patterned substrates were stored in a vacuum desiccator until used.

The multisine SPR Microchip as illustrated in FIG. 7A includes two 3-bit, 7-plug, continuous flow dual multiplug modulators integrated with SPR sensitive gold spots. Each modulator synthesizes a 28-level approximation to the multisine signal. This chemical signal generator chip was fabricated using a two-level PDMS stamp process. PDMS was molded over photolithographically patterned (Su-8 and AZ9260 for valve and flow layer molds respectively) Silicon wafers (4 inch diameter). The two-layer chip was then fabricated. The output flow channels (175×22 μm²) of the two modulators (sensing and reference) were aligned by the patterned gold spots as shown in right of FIG. 7B. The dual channel set-up was required to extract the binding signal from the bulk response recorded from the reference channel.

The gold spots were functionalized with PEG. Specifically, the gold spots were cleaned with 0.01 M HCL followed by thiolated cm-PEGylation with an underbrushed layer of a shorter m-PEG to minimize non-specific adsorption on the gold surface. This is followed by standard amine-coupling of ABS ligand using Sulfo-NHS (0.1 M) and EDC (0.4 M) to form a two-dimensional ligand immobilized sensing surface. The reference surface was blocked with ethanolamine in PBS buffer (25 mM sodium phosphate, pH 8.4) containing 0.01% SDS.

Phosphate-buffered saline (5 mM sodium phosphate, pH 7.4) was used as running buffer. Three different concentrations (A₀) of 5.2, 10.4 and 15.6 μM were used for the separate multisine excitation experiments, with later consisting of seven frequency components ranging from 2 mHz to 64 mHz. The channels were flushed with regeneration solution (0.1% SDS in running buffer) between the runs.

The kinetic constants kₐ and k_{d} for this ligand-ligate pair were obtained by plotting the observed pole frequencies p=2πfₚ obtained from the bode plot of the transfer function H(jω) for the three different concentrations of the analyte. The term fₚ here refers to the −3 dB point of the transfer function plots in the frequency (Hz) domain. In all the data sets, there exists a fixed imager intensity I_{offset} for all the imaging gold spots. This is first recorded at the beginning of the experiment and later on substracted from the measured signals to bring the baseline of the extracted signal to zero. The signal from the reference spots, I_{ref}, is then substracted from that of the sensor spots, Iₛₑₙₛₒᵣ, to obtain reference and sensor signals for further processing. The reference-sensor spot pairs are approximately at the same distance downstream from the either modulators and subtraction of their signals ensure elimination of the signal component from bulk response of analyte. The corrected time-domain responses from the multisine experiments then follow the simple relation I(t)=I₀·s(t) where I₀ is the proportionality factor, s(t) being the test signal.

FIG. 9A shows the time-domain response observed on a bare gold spot for a multisine signal of five discrete frequency components of {10, 20, 50, 100, 200} mHz with corresponding phases of {0, 0, π, 0, π}, generated by the two MPMs at a plug rate of 40 plugs/sec. This is equivalent to a single 28-level digitized output every 0.7 seconds. The multisine crest factor was 2.1. The analyte solution does not react with these non-functionalized gold spots such that the SPR response corresponds to the multisine spectrum of the test signal. This time-domain signal does not look particularly periodic, but when we calculate the power spectrum, we can clearly see the discrete frequency components of roughly the same power as shown in FIG. 9B.

This spectral method for characterizing the biochemical reaction using functionalized sensor and passivated reference gold spots was tested. A test multisine signal with minimized crest factor comprising of equally weighed seven binary weighted frequency components {1, 2, 4, 8, 16, 32, 64} mHz and corresponding phases {0, ⅔π, 5/3π, 5/3π, 5/3π, 5/3π, 0} was generated by the two MPMs. A time-domain plot of this analog signal is shown in FIG. 9C. The multisine waveform had a crest factor of 2.2. The average flow velocity was about 10 cm/s, which is sufficiently high to overcome mass transport effects for this configuration. The duration of the multisine run was ˜17 minutes corresponding to an input excitation synthesized with 40,000 plugs.

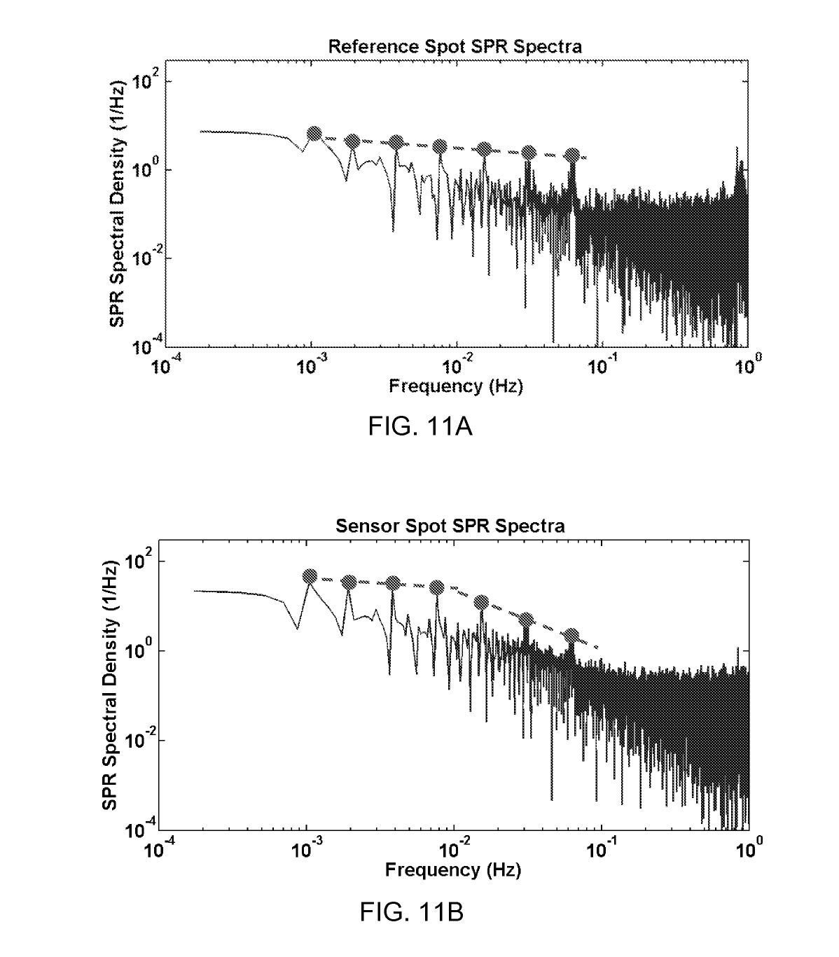

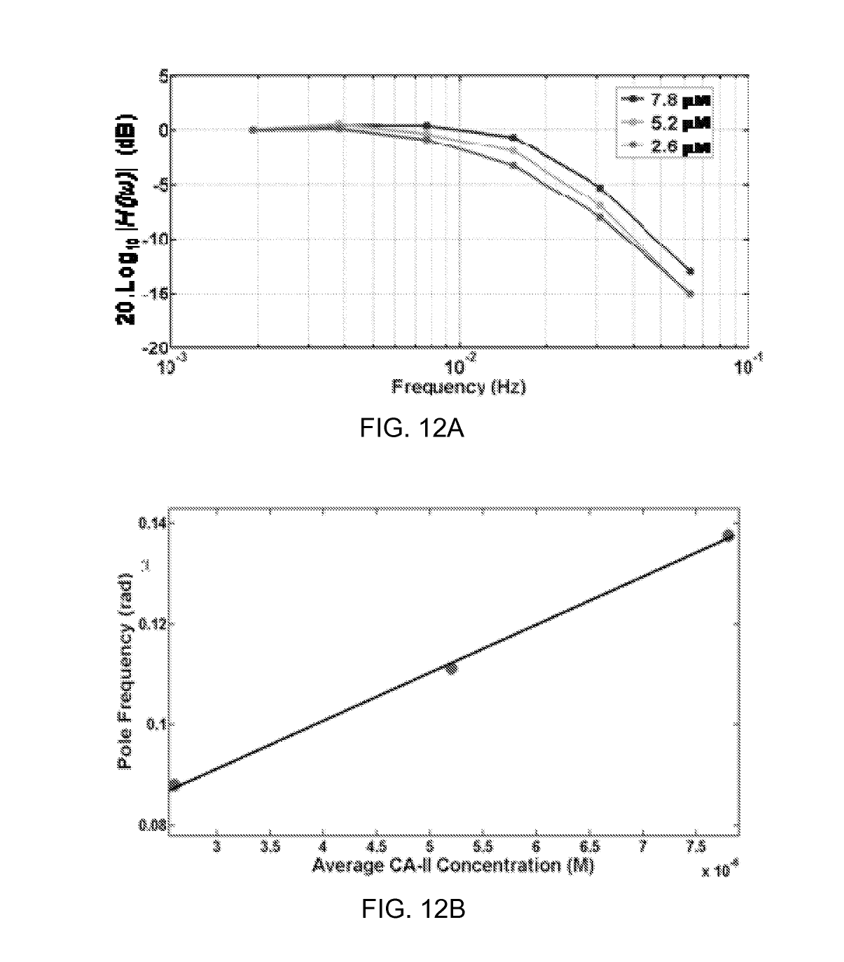

FIGS. 11A and 11B show the comparison of the SPR spectra under the multisine excitation of FIG. 9C for analyte concentration of 10.4 μM. Discrete frequency peaks in the spectra are clearly identifiable. The Fourier spectral density of the response from the non-reacting reference surface is relatively flat which is expected for a signal with components of equal weight, while the spectrum at the reactive sensor surface is attenuated at frequencies higher than the pole frequency. This shapes the spectra expected from the single pole roll-off behavior of the transfer function. The noise floor for all the components is about ten times lower than each of the frequency peaks. Hence, the response due to the input excitation signal can be clearly discriminated from the noise. The signal-to-noise ratio (SNR) for this multisine measurement is about 32 dB, which is about 7 dB higher than the corresponding single step-response method (SRM), while the overall measurement time is twice as long as required by SRM. In FIG. 12A, the magnitude of the transfer function is plotted versus the frequency to obtain a Bode plot for different analyte concentrations. The values of frequencies obtained from the −3 dB points for these three concentrations are then plotted versus their respective average concentrations. The pole frequency p follows a linear relation (y=m·x+c) with the analyte concentration. Accordingly, one can extract the kinetic constants kₐ and k_{d} from the slope m and y-intercept c of the linear fit respectively, as shown in FIG. 12B. The values for kₐ and k_{d} from this fit are found to be 9.5×10³ l/(M·s) and 6.2×10⁻² l/s, respectively. Therefore this method shows an ability of detection of low molecular weight species such as small drugs on large protein substrates.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A biosensor comprising:
a) a fluid flow channel including
i. an inlet to accept a fluid into the fluid flow channel;
ii. a binding sensor surface within the fluid flow channel including a fixed binding moiety on the binding sensor surface selected to bind with a complimentary target agent within the fluid to form a complimentary bound duplex; and
iii. an outlet;
b) a pulsing mechanism configured to drive the fluid into the fluid flow channel in a switched analyte flow; and
c) a binding response measurement mechanism associated with the binding sensor surface to measure surface binding responses from the binding sensor surface.

2. The biosensor of claim 1, wherein the fluid flow channel is a microfluidic channel.

3. The biosensor of claim 1, wherein the fluid flow channel has a channel length of about 0.1 mm to about 10 cm.

4. The biosensor of claim 1, wherein the fixed binding moiety is selected from the group consisting of ligand, protein, DNA, RNA, membrane and combinations thereof.

5. The biosensor of claim 1, wherein the complimentary target agent is selected from the group consisting of ligand, protein, DNA, enzyme, drug, membrane and combinations thereof.

6. The biosensor of claim 1, wherein the complimentary target agent has a mass less than 500 Da.

7. The biosensor of claim 1, wherein the pulsing mechanism includes a fast microfluidic valves.

8. The biosensor of claim 1, wherein the pulsing mechanism includes two interleaved multiplug pulse encoded modulators.

9. The biosensor of claim 1, wherein the switched analyte flow is pulsed at a frequency of 1 mHz to 10 Hz.

10. The biosensor of claim 1, wherein the switched analyte flow is a substantially constant flow rate.

11. The biosensor of claim 1, wherein the switched analyte flow is sinusoidal in analyte concentration.

12. The biosensor of claim 14, wherein the switched analyte flow is multisine in analyte concentration.

13. The biosensor of claim 1, wherein the binding response measurement system is a surface plasmon resonance measurement mechanism.

14. The biosensor of claim 1, wherein the binding response mechanism is an ellipsometric system, interferometric system, optical resonator system, a mechanical deflection system, a photonic crystal system, a calorimetric system, a surface acoustic wave system, or an electrochemical response system.

15. The biosensor of claim 1, wherein the binding response measurement mechanism further includes a Fourier transform module for processing the binding responses in frequency domain.

16. The biosensor of claim 1, further comprising analysis module configured to convert the binding responses to at least one binding property of the fixed binding moiety and the complimentary target agent.

17. The biosensor of claim 17, wherein the at least one binding property includes kinetic constants, concentration assay, and equilibrium constants.

18. The biosensor of claim 1, wherein the biosensor has a minimum detectable mass of 10 Da or less.

19. A biosensing method, comprising:
a) passing a fluid across a binding sensor surface using a switched analyte flow, said binding sensor surface including a fixed binding moiety and the fluid including a complimentary target agent;
b) measuring binding responses during contact of the fluid with the binding sensor surface; and
c) analyzing the binding responses using frequency domain analysis to identify at least one binding property between the fixed binding moiety and the complimentary target agent.

20. The method of claim 24, wherein the measuring is based on surface plasmon resonance and measurement as a function of at least one of resonant angle, resonant frequency or polarization.

21. The method of claim 24, wherein the switched analyte flow is feedback locked using the surface plasmon responses.

22. The method of claim 24, wherein the switched analyte flow is sinusoidal.

23. The method of claim 27, wherein the switched analyte flow is multisine.

24. The method of claim 24, wherein the switched analyte flow is variable and analyzed using linear transforms.

25. The method of claim 29, wherein the linear transforms are chirp or wavelet transforms.

26. The method of claim 24, wherein the switched analyte flow is a substantially constant flow rate.

27. The method of claim 24, wherein the measuring is based on at least one of interferometry, ellipsometry, reflectance, mechanical deflection, oscillator mass loading, optical resonance, optical waveguide, photonic crystal, calorimetry, surface acoustic wave, and electrochemical responses.
